(19) United States
(12) Patent Application Publication
(10) Pub. No.: US 2017/0132391 A1
(43) Pub. Date: May 11, 2017

(54) MEDICAL DEVICE COMMUNICATION

(71) Applicant: MICRODOSE THERAPEUTX, INC., Ewing, NJ (US)

(72) Inventor: Mark Steven Morrison, Basking Ridge, NJ (US)

(73) Assignee: MICRODOSE THERAPEUTX, INC, Ewing, NJ (US)

(21) Appl. No.: 15/318,594

(22) PCT Filed: May 20, 2015
(86) PCT No.: PCT/US2015/031686

§ 371 Date: Dec. 13, 2016

Related U.S. Application Data
(60) Provisional application No. 62/011,808, filed on Jun. 13, 2014.
(60) Provisional application No. 62/135,798, filed on Mar. 20, 2015.

(51) Int. Cl. G06F 19/00; A61M 15/00

(52) U.S. Cl. CPC G06F 19/3456; A61M 15/0065; A61M 15/0051; A61M 15/0026; A61M 2205/52; A61M 2205/502; A61M 2205/581; A61M 2205/3584; A61M 2205/3553; A61M 2202/064; A61M 2205/3334

(57) ABSTRACT

Disclosed herein is a method for communicating between a medical device 101 and an electronic device 103, the method comprising: establishing 203 communications between a medical device 101 and an interface device 102; and establishing 205 wired communications between the interface device 102 and an electronic device 103, such that data can be communicated between the medical device 101 and the electronic device 103 via the interface device 102; wherein the wired communications is provided between a port of the interface device 102 and a port of the electronic device 103. Advantageously, an interactive patient management system is realised in a convenient and inexpensive manner.

### CROSS REFERENCE TO RELATED APPLICATIONS

This application claims priority from U.S. Provisional Application No. 62/011,808, filed Jun. 13, 2014, and from U.S. Provisional Application No. 62/135,798, filed Mar. 20, 2015. The disclosures of each of these applications are incorporated herein by reference in their entirety for all purposes.

### FIELD OF THE INVENTION

The present invention relates generally to communications with medical devices. In particular embodiments of the invention provide an interface device that supports communications between a medical device and an electronic device. Advantageously, an existing household electronic device, such as a digital television, can be used by a patient for improved interaction with a medical device without an Internet connection being required.

### BACKGROUND TO THE INVENTION

Enabling a medical device with wireless communications results in an improved user experience both for patients and for medical practitioners. It is known for large medical devices to be able to wirelessly communicate, using Bluetooth, to an Internet router and then use the Internet to retrieve relevant data from a remote server. Such a system may be installed in hospitals for large medical devices in specialist medical treatment environments.

A problem experienced by known implementations of systems in which medical devices are enabled with data communications, is that it is difficult and expensive to implement such systems in a user's home. Even if it is possible for the medical device to be used outside of a hospital, the user's home needs to have the required communications infrastructure. A user may therefore be required to purchase a suitable wireless router, and other equipment, that the user would not otherwise need. Known communications techniques with the medical device also require a functioning Internet connection and this may not be reliable or always accessible. In addition, the user is required to have an Internet enabled electronic device, such as a smart phone, tablet or PC, in order to display retrieved information from the remote server. The display of data to a user also requires the device to be already turned on and for the electronic device to be executing a suitable application.

Accordingly, there is a general need for providing a convenient and inexpensive technique for improving the interaction of a user with a medical device and engaging a patient in their therapy.

### SUMMARY OF THE INVENTION

According to a first aspect of the present invention, there is provided a method for communicating between a medical device and an electronic device, the method comprising: establishing communications between a medical device and an interface device; and establishing wired communications between the interface device and an electronic device, such that data can be communicated between the medical device and the electronic device via the interface device; wherein the wired communications is provided between a port of the interface device and a port of the electronic device.

Preferably, the port of the interface is an High Definition Multimedia Interface, HDMI, port and the port of the electronic device is an HDMI port.

Preferably, the communications between the medical device and the interface device are wireless communications.

Preferably, Bluetooth is used for the wireless communications.

Preferably, the method further comprises transmitting data from the medical device to the electronic device via the interface device; and displaying data dependent on the transmitted data on a display of the electronic device.

Preferably, the method further comprises sending, by the interface device, an instruction to the electronic device to switch on.

Preferably, the method further comprises sending, by the interface device, a message to the electronic device; and displaying the message on a display of the electronic device.

Preferably, the data sent to the electronic device by the interface device is generated by the interface device.

Preferably, the data sent to the electronic device by the interface device is generated by the medical device and sent to the interface device by the medical device.

Preferably, the method further comprises the medical device wirelessly transmitting output data to a printer and/or to an Internet router.

Preferably, said output data is transmitted from the medical device using Bluetooth.

Preferably, the method further comprises the interface device wirelessly transmitting output data to a printer and/or to an Internet router.

Preferably, said output data is transmitted from the interface device using WiFi.

Preferably, the interface device comprises a first wireless interface for Bluetooth communications and a second wireless interface for WiFi communications.

Preferably, the method further comprises executing one or more computer programs by the interface device for controlling the medical device and/or electronic device.

Preferably, the method further comprises: a user interacting with the electronic device; and the electronic device communicating data dependent on the user interaction to the interface device.

Preferably, the method further comprises: obtaining, by the medical device, real-time data that is dependent on the use of the medical device by a user; transmitting the obtained data to the electronic device via the interface device; and displaying, on a display of the electronic device, data dependent on the obtained data such that the electronic device displays substantially real-time data dependent on the use of the medical device.

Preferably, the electronic device is a television.

Preferably, the medical device is an inhaler.

Preferably, the medical device is a tidal inhaler.

According to a second aspect of the invention there is provided an interface device configured to communicate with a medical device and an electronic device according to the method performed by an interface device as set out for the first aspect.

According to a third aspect of the invention there is provided a system comprising a medical device, interface device and electronic device, wherein the medical device and electronic device are configured to communicate with each other via the interface device according to the method of the first aspect.

### BRIEF DESCRIPTION OF DRAWINGS

Embodiments of the present invention will now be described, by way of example only, with reference to the accompanying drawings, in which:

FIG. 1 shows a system according to an embodiment; and

FIG. 2 is a flowchart of a method according to an embodiment.

FIG. 3 shows a menu of user options that is displayed on a television screen according to an embodiment.

FIG. 4 shows the more specific applications/functions that may be selected through selection of any of the options shown in FIG. 3.

### DESCRIPTION OF EMBODIMENTS

Embodiments solve at least some of the above-identified problems by providing a convenient and inexpensive technique for communicating between a medical device 101 and an electronic device 103 without a functioning Internet 106 connection being required.

Embodiments are particularly appropriate for small portable medical devices 101. It is not generally known for small medical devices 101 to be capable of wireless communications. However, research in this area is increasing and a suitable such handheld, battery operated, inhaler is being developed by TEVA®.

The electronic device 103 is used to display data related to the medical device 101 and, preferably, interact with the user.

An interface device 102 is provided for supporting communications between the medical device 101 and the electronic device 103. The interface device 102 preferably has a wireless communications interface for communicating with the medical device 101 and a port for a wired connection to an electronic device 103.

Embodiments are also particularly appropriate for use with any electronic device 103 with an High Definition Multimedia Interface, HDMI, port, such as an HDMI enabled television. When the last analog television broadcasts were transmitted on Jun. 12, 2009, the original analog television set became largely obsolete, unless connected to some form of digital cable or satellite box. This spurred the development of replacement digital televisions incorporating an HDMI port. Many of these digital televisions also incorporate Wi-Fi, USB and memory card options and provide an advanced multi-media experience. Users can use such televisions to watch programs, display photographs, play videos and access the Internet 106. A lot of households have some form of digital television, or analogue television that is being operated through a digital-to-analogue converter, that is an HDMI enabled electronic device 103. Embodiments therefore improve a user's interaction with a medical device 101 by providing communications between the medical device 101 and the electronic device 103. Advantageously, the system is convenient and inexpensive to implement since an Internet 106 connection is not required and an existing HDMI television may be used. Embodiments realise an interactive patient management system in a user's home. For example, the system may be used to display medical records, dosage regime information, or reminder messages on a user's television and the television's remote control may be used to control the printing of medical records and other functions.

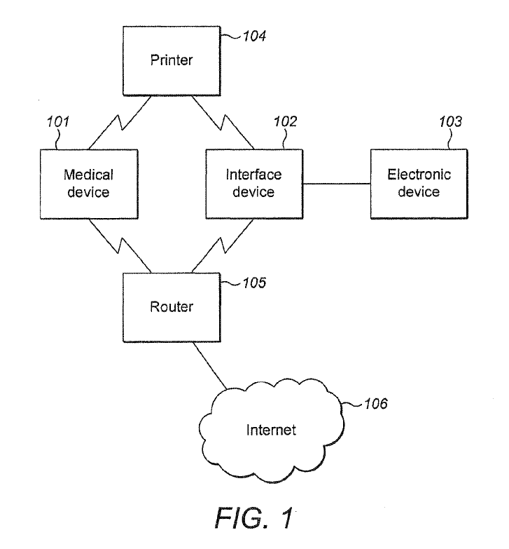

FIG. 1 shows a system according to an embodiment. The system comprises at least a medical device 101, an interface device 102 and an electronic device 103. The system may also comprise a router 105, that is connected to the Internet 106, a printer 104 and other auxiliary devices (not shown in FIG. 1).

The medical device 101 may be any type of medical device 101 capable of data communications. Preferably, the medical device 101 is a small, handheld, battery operated, medical device 101 such as an inhaler.

Preferably, the medical device 101 is a tidal inhaler, such as a dry powder tidal inhaler.

Preferably, the dry powder tidal inhaler includes a piezo vibrator for aerosolising the dry powder. The dry powder tidal inhaler may contain dry powder medicament within blisters on a blister strip. In use, the blister strip may be advanced and opened such that the medicament may be transferred from the blister to a dosing chamber via a short passageway, the dosing chamber being in close proximity to or in contact with the piezo vibrator within the device. The dose chamber also having one or more apertures opposite the piezo to allow ejection of the powder into a passageway in contact with a mouthpiece or nosepiece for patient inhalation. The medical device 101 may be a nebulizer, such as a dry powder nebulizer. Preferably, the medical device 101 is an inhaler that comprises a flow sensor for monitoring a user's breathing. Actuation of the piezo and thus ejection of the medicament from the dosing chamber for inhalation by the patient may be modulated by signals generated by the flow sensor and/or other patient activated controls.

Alternatively, the device may for example be a dry powder inhaler including a mouthpiece for patient inhalation, a delivery passageway for directing an inhalation induced air flow through the mouthpiece, a channel extending from the delivery passageway, and a reservoir for containing medicament, with the reservoir having a dispenser port connected to the channel, and also including a cover which is pivotally mounted to the case of the inhaler for covering the mouthpiece and for actuating dispensing of medicament from the reservoir via a hopper. The inhaler may have a breath-actuated mechanism where inhalation by the patient induces delivery of the metered medicament.

Thus, the medical device 101 may for example be based on the medical devices as disclosed in any of US 2011/0162642A1, U.S. Pat. No. 8,439,033B2, US 2011/0000481A1, U.S. Pat. No. 7,318,434B2, WO 02/00281, WO 01/097889 and/or WO 2005/034833, with the device further enabled for data communications according to embodiments. Thus, the medical device 101 may be based on a MicroDose® device or Spiromax®/DuoResp®Spiromax® device, manufactured by Teva®, with the device further enabled for data communications according to embodiments.

The medical device 101 is enabled with one or more interfaces for data communications. The medical device 101 comprises electronic processors, memory and other electronics for generating data for transmission from the medical device 101. The transmitted data may be, for example, stored data on the medical device 101, such as a dosage regime for a medicine dispensed by the medical device 101, or data generated by the medical device 101 in dependence on the use of the medical device 101 by a patient.

The data communication from the medical device 101 is preferably by wireless communications, as shown in FIG. 1, but may alternatively be over a wired data connection. The wireless communications are preferably implemented using Bluetooth, in particular Low Energy Bluetooth. Bluetooth is preferable since it can transmit data at a low power. This is advantageous for communications with the medical device 101 as it is important to have low power demands on its battery. Other forms of wireless communications, such as WiFi, provide a larger bandwidth than Bluetooth but require more power. However, because the amount of transmitted data from the medical device 101 will typically be small, such as the data within a medical record or a single picture, the bandwidth provided by Bluetooth is sufficient.

The interface device 102 is configured to communicate with both the medical device 101 and the electronic device 103. The interface device 102 can be configured to provide an interface between the medical device 101 and the electronic device 103 so that data received by the interface device 102 from the medical device 101 is communicated to the electronic device 103 and vice versa.

The interface device 102 preferably comprises a wireless communications interface for supporting wireless communications with the medical device 101. For the reasons provided above, the wireless communications between the medical device 101 and the interface device 102 are preferably implemented using Bluetooth. The interface device 102 may also support other types of wireless communications, in addition to Bluetooth, such as WiFi. The WiFi, or other types of wireless communications, may be transmitted over the same wireless interface as used for Bluetooth communication or over one or more separate wireless interfaces. The communications between the interface device 102 and the electronic device 103 are preferably over a wired connection. The port on the interface device 102 for supporting the wired connection may be any type of suitable port. Preferably, the interface device 102 comprises an HDMI port for the data communication with the electronic device 103. The interface device 102 may also be configured to communicate with any other devices that may be present in the system, such as the router 105 and printer 104 shown in FIG. 1. Preferably, the communications with the other devices are wireless and implemented by Bluetooth and/or WiFi communications.

The interface device 102 preferably is a computing device that comprises one or more memories and processors for executing one or more computer programs. The one or more computer programs may support, or enhance, the data communications between the medical device 101 and the electronic device 103. Alternatively, or in addition, the one or more computer programs may configure the interface device 102 to control the medical device 101, electronic device 103, and/or any other devices that the interface device 102 is communicating with.

A known device that may be used to implement the interface device 102 is an Android® mini PC, known as Cotton Candy®. This is particularly preferable as it is a small handheld computing device that is capable of wireless communications using Bluetooth and WiFi, as well as wired communications via HDMI and USB ports. The interface device 102 is preferably an HDMI Dongle.

The known use of the Cotton Candy® device is for improving security by providing a separate computer for business uses. A computer may be used normally for personal use. For business use, the Cotton Candy® device is connected to the computer and the Cotton Candy® device operates through the computer. This prevents any malware that may be picked-up through the personal use of the computer from affecting business uses. It is not known for the Cotton Candy® device to be used for the specific purpose of an interface to a medical device 101. With regard to known devices for providing an interface from wireless communications to an HDMI port, it is only known to implement this with WiFi as this is the only way of providing sufficient bandwidth for full HDMI capabilities, e.g. streaming a high definition live video stream from the Internet 106 to a digital television. Interfacing between an HDMI port and Bluetooth communications is not known, or obvious, as Bluetooth does not provide the bandwidth required for using the HDMI port to its full potential.

The electronic device 103 is configured to communicate with the interface device 102. The communications between the interface device 102 and the electronic device 103 are preferably over a wired connection. The port on the electronic device 103 for supporting the wired connection may be any type of suitable port. Preferably, the interface device 102 comprises an HDMI port for the data communication with the electronic device 103. The electronic device 103 comprises a screen for displaying information. The electronic device 103 may also be configured to communicate with peripheral devices, such as remote control of the electronic device 103.

The electronic device 103 may be any suitable type of electronic device 103. Preferably, the electronic device 103 is a television enabled with an HDMI port. This may be a digital television with an integral HDMI port or an analogue television operated through a digital-to-analogue converter that is connected to the interface device 102.

Other devices may also be present in the system, such as the router 105 and printer 104 shown in FIG. 1. These are preferably configured to communicate with the medical device 101 and/or the interface device 102. The communication is preferably over a wireless data connection, such as WiFi or Bluetooth.

Advantageously, the system according to embodiments allows communication between a medical device 101 and an electronic device 103 over a local network within a user's home. There is no need for the home to have a functioning Internet 106 connection or wireless router 105. The electronic device 103 may be an existing HDMI enabled television in which case the only new equipment required is the interface device 102.

The interface device 102 is also a computing device and this allows an advanced interactive patient management system to be realised. Some of the possible system implementations and functions according to embodiments are described below.

The medical device 101 may generate medical data by recording data when in use. For example, an inhaler may record when it is being used, the dosage of medicine dispensed during each use, measured battery levels of the medical device 101 and other data. The medical device 101 may also store medical data, and any other type of relevant data, such as instructions for use of the medical device 101, a prescribed dosage regime, medical records, general advice, etc. The interface device 102 may also store medical data, such as that described above, as well as other types of data. The data may be communicated to the electronic device 103 and displayed on a display of the electronic device 103.

The system preferably comprises a printer 104. A user may then choose to print some, or all, of the currently displayed data by the electronic device 103 or stored data within the medical device 101 or interface device 102. The printed data can then be reviewed by a patient in person with a doctor.

The communication of data between the medical device and the electronic device 103, and the input of user instructions into the system, may be in response to an instruction received by the electronic device 103, such as a user using a television remote control to select an option from a displayed menu, or a direct input to the medical device 101 or interface device 102, such as a user pressing a button on the medical device 101 or interface device 102.

The interface device 102, and electronic device 103, may have a larger memory than the medical device 101 and data generated by the medical device 101 may be automatically transmitted from the medical device 101 for storage on either the medical device 101 or the electronic device 103 in order to reduce the storage requirements on the medical device 101.

An advantage of the electronic device 103 being an HDMI enabled television is that the HDMI standard has a feature known as Consumer Electronic Control, CEC, that provides useful functionality for suitably enabled televisions.

Preferably, the interface device 102 uses CEC to control the television. The interface device 102 may turn the television on, adjust its volume, display text messages over existing programming and other functions. For example, a patient could be reminded to take a dose by an on screen prompt while the patient is watching a broadcasted television program. This particular feature is known to as ‘On Screen Display over CES’. If the television happened to be switched off at the time a dose was due, the interface device 102 could turn it on and display the message and/or play an audio message that a dose is due. By similar means, it is possible to provide reminders about prescription refills or upcoming doctor's appointments. It is also possible to record information to a DVR connected to the television, for later review.

In this context, the user could interact with the medical device 101 in a very similar way to how they already interact with their television and/or DVR. The television, or DVR, remote control could be used to page through data, displayed by the electronic device 103, and stored on the interface device 102, electronic device 103, or DVR, to obtain medical data or any other type of relevant data.

Preferably, the interface device 102 stores videos that can be played on the electronic device 103. The videos may demonstrate proper use of the device, or provide troubleshooting information presented in an interactive fashion. Such data could be pre-programmed into the interface device 102 at the time of its manufacture or issue.

It is particularly preferable for the medical device 101 to be an inhaler that has flow monitoring capabilities so that a real-time display of a patient breathing can be generated on the electronic device 103. This has training and diagnostic purposes and a patient can be provided with real-time coaching while using their inhaler and encouragement to assist them in getting through their treatment.

Although not required for realising embodiments, communicating from the interface device 102 to a router 105 with a functioning internet 106 connection allows additional advantageous features, such as including remote patient monitoring and diagnostics, real-time help for patients having difficulty using their device, the ability for doctors to monitor a dose in progress, and a broadcasting channel for the distribution of more detailed information.

Accordingly, embodiments provide an inexpensive and simple technique for creating an interactive patient management system over a local area network. The system preferably uses an HDMI television to interact with a user. Since a user may already own such a television, there are significant cost benefits. In addition, it is not necessary for the system to have a router 105 or functioning Internet 106 connection. This both increases the reliability of the system and there are also security benefits as the medical data of a patient is maintained within the local network and not transmitted over the Internet 106.

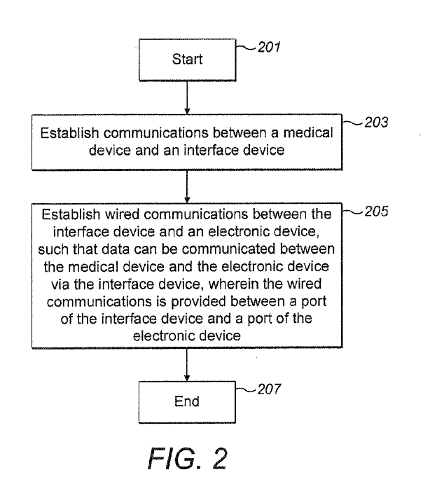

FIG. 2 is a flowchart of a method according to an embodiment.

In step 201, the process begins.

In step 203, communications are established between a medical device 101 and an interface device 102.

In step 205, wired communications are established between the interface device 102 and an electronic device 103, such that data can be communicated between the medical device 101 and the electronic device 103 via the interface device 102, wherein the wired communications is provided between a port of the interface device 102 and a port of the electronic device 103.

In step 207, the process ends.

The interface device 102 according to embodiments allows an interactive patient management system to be set-up in a patient's home. Preferably, the interface device 102 is a home health hub that is able to support wired and wireless communication with a plurality of other devices. The system is preferably performed in accordance with the IEEE 11073 family of standards so that any devices compliant with these standards are supported. The combined operation of the home health hub with other devices creates a rich multimedia experience directly related to a user's, i.e. patient's, therapy.

For example, videos may be played on a television screen in response to a patient using a device, the video interacting with the patient in the same way that a personal assistant would. Television displays are typically large and they are therefore particularly advantageous for use as a primary visual interface with a patient. In particular, elderly patients tend to struggle with reading small display screens, such as those found on mobile telephones. The cost of digital televisions is decreasing and it will not be long before most homes would be expected to have a digital television compatible with an interface device 102 according to embodiments, such as an HDMI dongle.

Preferably, the home health hub results in patients being engaged directly, without the patient being required to do anything. For example, the first time a patient turns on their television in the morning, the interface device 102 could detect this and respond with a friendly greeting displayed on the television screen. This could be followed by a reminder message about an upcoming prescription refill, or the need to charge the battery of one of their medical devices 101. Similar messages can be displayed to a patient in the scenario of a patient already watching a television program. Preferably the television is also configured to turn on automatically when a patient uses one of their medical devices 101. Preferably patients with poor vision could have information displayed in large font, or communicated by synthesized voice messages.

Embodiments greatly improve home health care. As far as possible, most patients prefer to support themselves without being a burden on others. However, people responsible for the patient need to know if the patient is taking their medication and using their medical devices 101 properly. An interactive patient management system meets the above requirements of a patient and those responsible for the patient.

For example, consider the situation of an inhaler interacting with a patient through their television. A patient opening the cap on their inhaler could then be presented with a message on their television, or hear a pleasant voice instructing them what to do next. This provides desirable and comforting feedback to patients who otherwise may be uncertain about how to use their device properly.

As another example, when a patient takes their medication, a message could appear on their television telling the patient that the dose was delivered. This relieves any anxiety that may occur when taking medications from which no direct feedback exists from taste or other sensations.

Playing a video on the television may improve the interaction with a patient than if a message is displayed. For example, if a patient isn't quite reaching proper inspiratory flow to receive medication from an inhaler, the inhaler could detect this low flow condition, send a message to the interface device 102 and, in dependence on the message, a video could then be played on the television showing proper technique. Alternatively, or in addition, a real-time display of the effort exerted by the patient compared to what is needed for proper therapy could be displayed. Such feedback could be unique to each type of medical device 101, giving patients an experience that is tailored to their specific situation. By providing feedback specific to particular patients and their devices, including whether or not a patient has used a device properly, it is easier for a patient to be able to live independently. In addition, if a patient comes to associate their ability to live independently with a product that is simple to use and requires no special involvement on their part, they are more likely to achieve a higher level of adherence to their medical treatment.

An interactive patient management system therefore results in a patient effectively being provided with a virtual personal assistant that helps patients live independently and also live better.

However, even with such feedback, some patients may still be reluctant to use a system that monitors them. There is therefore a need for some way of engaging the patient that goes beyond simple reminders and in-use feedback. Since many diseases are chronic in nature, the patient may need to be engaged for a very long time. An embodiment meets these requirements by incorporating gaming into the system. Large screen televisions already play a major role in the popular video game market. They could therefore support games that interact with the patient in ways that encourage adherence as well as compliance with a medical treatment. For example, games may inherently encourage proper breathing techniques or device usage. A simple flow meter may be displayed on a television showing some target flow to be achieved. The patient flow could be shown as a cursor that moves in response to inspiratory flow. The game could be as simple as moving the cursor toward the target, or chasing a moving target. Such games could even develop or maintain cognitive skills. The games may also include a social media component, where players communicate while playing against one another. Having such social interaction may encourage adherence by giving people something to look forward to, effectively engaging them in the use of their device. Patients using a specific medical device 101 could be connected to other users of the same type of device, forming a kind of support group. In this manner, patients can enjoy a community experience, while at the same time reaping the benefits of good adherence.

According to another embodiment, the interactive patient management system may communicate recorded data to a remote server such that drug usage is tracked in real-time. Such information could be used to monitor the progression of certain diseases across the globe or identify emerging health patterns relevant to public health.

Preferably, the medical device 101 can be controlled by a television remote control via the interface device 102. This is particularly advantageous for medical devices 101 without a display (e.g. pill containers) or medical devices 101 with a limited user interface (e.g. inhalers). By using the television remote control, it would be possible to program user preferences, dose reminders, and even patient metrics required for lung function (e.g. age, height, weight) all on the big screen. The television preferably displays a menu that allows the user to program their medical device 101 and thereby personalise their experience.

Alternatively, interface device 102 may be provided with a remote control for direct communication between the remote control and the interface device 102. The remote control could be used to program the medical device 101 as described above.

The interface device 102 may also be a television dongle. Television dongles are carried by users so that users can access their favourite pay-per-view television subscriptions (e.g. Google Play or Amazon Prime) while travelling. A user just plugs the dongle into an open port of their hotel television, completes an Internet connection, and then starts watching their favourite programs as if they were in their own living room.

The portable nature of the interface device 102 means that patient support is still provided to patients whilst they are traveling and this can fit in with their normal routine. Patients may even bring the interface device 102 to their doctor's office, where it could be plugged into a television, or the doctor's computer, to retrieve the patient's medical data.

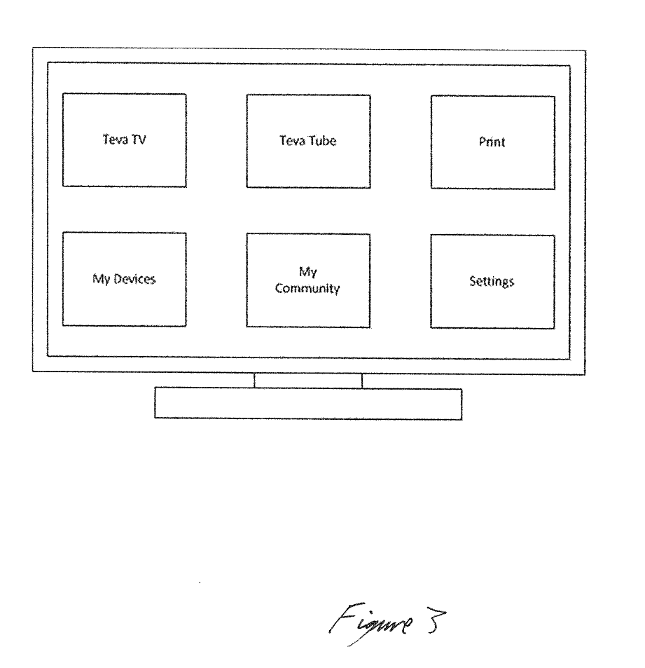

FIG. 3 shows a menu of user options that is displayed on a television screen according to an embodiment. The options are displayed in response to activation of the user interface device 102, such as due to it being plugged into a television or the television being turned on.

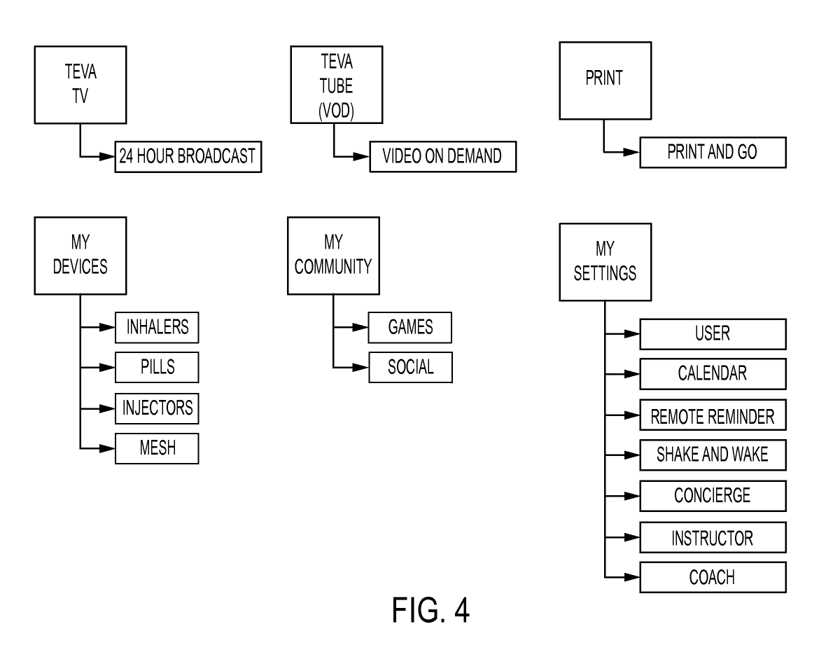

FIG. 4 shows the more specific applications/functions that may be selected through selection of any of the options shown in FIG. 3.

Using a remote control, users can navigate menus and customize the level of interaction they want with their medical devices 101. For example, if a user has two medical devices 101, that are both supported by the same interface device 102, and the user wants to be coached while using only one of the medical devices 101, the user is able to enable the coaching option for one of the medical devices 101 only. As another example, if a user would like training in the proper use of their medical devices 101, this is a function that can be selected as well.

With regard to the applications demonstrated in FIG. 3, ‘Teva TV’ is an example of a service that delivers video to patients on a 24 hour basis. Separate 30 minute program loops could be created for different channels, one for each disease type. Each channel could be automatically selected based upon the types of medical devices 101 registered to the user of the interface device 102. Patients would then only see health information associated with the medications/diseases they have. This also provides easy access to professionally produced videos that may already be available on the Internet but not necessarily easy to find.

‘Teva Tube’ would be a Video-On-Demand (VOD) service where patients can select the health related programs (i.e. videos) they wish to view. The programs available for selection could be automatically based upon the types of devices registered to the user of the interface device 102. Patients would only see health information associated with the medications/diseases they have. This also provides easy access to professionally produced videos that may already be available on the Internet but not necessarily easy to find.

‘Print’ would be selected by the patient to print out data, such as usage history, associated with their medical devices 101. The printout could then be shared with a doctor as a hardcopy to be stored in the patient files. This function can be used without an Internet connection and facilitates sharing of hardcopy data which minimises concerns about cybersecurity.

Selecting the ‘My Devices’ tab brings up a list of all devices currently known (i.e. registered) with the interface device 102. Selecting specific devices within that tab could reveal specific information about those devices. For example, an inhaler could show the number of doses remaining and/or a chart showing usage history by day, week or month.

By selecting the ‘My Community’ tab, users can access various games and social media. Games could engage the patient individually or with other users using the same type medical device 101. Games could be associated with the types of medical devices 101 currently known (i.e. registered) by the interface device 102. Social media would provide anonymous access to other users of the same type medical device 101, giving them access to others with similar health issues.

By selecting the ‘My Settings’ tab, users could see a list of all the ways to configure their medical device(s) 101. For example, users could enter the date and time, set dose reminders, and possibly even reminder ‘ringtones’. Preferably, users enter a unique screen name for use in games and social media. That same name is then used in personal greetings displayed to the patient by the television.

Patients could also enable or disable features individually for any medical devices 101 known to the interface device 102. Such features may include: Remote Reminder, Shake and Wake, Virtual Concierge, Virtual Instructor and Virtual Coach.

There are many scenarios in which patient care and experience is greatly improved by the interactive patient management system according to embodiments. Some examples are described below.

A patient may be reminded to take a dose by a sound emitted from their inhaler. However, the reminder is not heard by the patient due the patient watching television in a different room. The inhaler also sends a message to the patient's television which sends a reminder that the patient receives.

A patient may forget which pills he/she has taken. To find out, the patient simply shakes the pot of pills. In response to detecting the shaking, the pot of pills sends a message to the patient's television and the patient can determine which pills have been taken from the information obtainable from interaction with the television.

Medical devices 101 may detect if they need to be refilled. A message may be automatically displayed to a user that prompts the user to order a refill for a medical device 101.

A patient is not confident when using their inhaler. The patient turns on their television as is shown by an instructional video.

A patient may want guidance when using an inhaler. The patient may activate a coaching feature which receives real-time data from a sensor inside the inhaler and synchronises an instructional video to monitored use of the inhaler. The patient can then be provided with real-time feedback.

The configuring of medical devices 101, and programs provided by the interface device 102, need not be by the patient but may be by a doctor, carer or relative. The configuration may provide personal data, such as a patient's age, to help detect medical conditions and/or set dosage reminders.

Advantageously, the interactive patient management system according to embodiments actively engages patients in their therapy, immersing them in a rich multimedia experience. The television is a particularly advantageous device for user interaction due to the large size of the display screen and its reliable nature. Such advantageous user interaction would be a lot worse with, for example, a smart phone that has a small display screen and is unreliable due to its dependence on battery power.

Embodiments also include a number of modifications and variations to the above-described techniques.

The system shown in FIG. 1 may comprise a plurality of medical devices 101 and the interface device 102 configured to support communications with, and control of, each of the plurality of medical devices 101.

Although communication between the interface device 102 and the electronic device 103 is preferably via HDMI ports on each device, the communication may alternatively be via a USB ports or other types of ports. The communication between the interface device 102 and the electronic device 103 may alternatively be by wireless communications.

The communication between the interface device 102 and a medical device may be via a wired connection that is preferably provided in addition to the wireless connection capabilities of the interface device 102.

The electronic device 103 may be any suitable type of electronic device 103, such as a computer.

The transmission of some, or all, of the data between any of the medical device 101, interface device 102 and electronic device 103 may be performed automatically and not necessarily in response to a user instruction.

The interface device 102 may also use Bluetooth or WiFi to communicate with a user's smart phone in order to display notifications to the user and to receive instructions from the user.

The interface device 102 may be provided by a user's smart phone. The smart phone preferably communicates with the medical device 101 by Bluetooth communications and with the electronic device 103 by WiFi. The electronic device 103 may be a WiFi enabled television.

A smart phone may execute one or more Apps so that it is configured for use in the medical system.

The flow chart and description thereof herein should not be understood to prescribe a fixed order of performing the method steps described therein. Rather, the method steps may be performed in any order that is practicable. Although the present invention has been described in connection with specific exemplary embodiments, it should be understood that various changes, substitutions, and alterations apparent to those skilled in the art can be made to the disclosed embodiments without departing from the spirit and scope of the invention as set forth in the appended claims.

1-22. (canceled)

23. An interface device for communicating with a medication delivery device and an electronic device, the interface device comprising:
a memory; and
a processor configured to:
receive medication delivery data from the medication delivery device;
determine a program related to the medication delivery data, the program being associated with a graphic or an audio file; and
send a control message to the electronic device to display the graphic file on a display of the electronic device or play the audio file via a speaker of the electronic device.

24. The interface device of claim 23, wherein the program is stored in the memory of the interface device.

25. The interface device of claim 23, wherein the program is stored in a remote server, the processor further configured to obtain the program from the remote server.

26. The interface device of claim 23, wherein the medication delivery data is associated with a patient's usage of the medication delivery device.

27. The interface device of claim 26, wherein the medication delivery device comprises an inhaler and the medication delivery data comprises inhalation data.

28. The interface device of claim 23, wherein the graphic or the audio file comprises a dosage regimen or a reminder message, wherein the dosage regimen or the reminder message is associated with the medication delivery data.

29. The interface device of claim 23, wherein the program comprises an instruction for a proper use of the medication delivery device.

30. The interface device of claim 23, wherein the processor is further configured to receive the medication delivery data in real-time, determine the program in real-time, incorporate the medication delivery data in the program in real-time, and send the control message to the electronic device in real-time.

31. The interface device of claim 31, wherein the program comprises a game that engages a patient who uses the medication delivery device.

32. A method for communicating with a medication delivery device and an electronic device, the method comprising:
receiving medication delivery data from the medication delivery device;
determining a program related to the medication delivery data, the program being associated with a graphic or an audio file; and
sending a control message to the electronic device to display the graphic file on a display of the electronic device or play the audio file via a speaker of the electronic device.

33. The method of claim 32, further comprising storing the program in a memory of an interface device.

34. The method of claim 32, wherein the program is stored in a remote server, the method further comprising obtaining the program from the remote server.

35. The method of claim 32, wherein the medication delivery data is associated with a patient's usage of the medication delivery device.

36. The method of claim 35, wherein the medication delivery device comprises an inhaler and the medication delivery data comprises inhalation data.

37. The method of claim 32, wherein the graphic or the audio file comprises a dosage regimen or a reminder message, wherein the dosage regimen or the reminder message is associated with the medication delivery data.

38. The method of claim 32, wherein the program comprises an instruction for a proper use of the medication delivery device.

39. The method of claim 32, further comprising:
receiving the medication delivery data in real-time;
determining the program in real-time;
incorporating the medication delivery data in the program in real-time; and
sending the control message to the electronic device in real-time.

40. The method of claim 39, wherein the program comprises a game that engages a patient who uses the medication delivery device.

41. An interactive patient management system comprising:
a medication delivery device configured to determine and send medication delivery data associated with a usage of the medication delivery device; and
an interface device for communicating with the medication delivery device and an electronic device, the interface device configured to:
receive the medication delivery data from the medication delivery device;
determine a program related to the medication delivery data, the program being associated with a graphic or an audio file; and
send a control message to the electronic device to display the graphic file on a display of the electronic device or play the audio file via a speaker of the electronic device.

42. The interactive patient management system of claim 41, wherein the interface device comprises a memory, and the program is stored in the memory of the interface device.

43. The interactive patient management system of claim 41, wherein the program is stored in a remote server, and the interface device is further configured to obtain the program from the remote server.

44. The interactive patient management system of claim 41, wherein the medication delivery data is associated with a patient's usage of the medication delivery device.

45. The interactive patient management system of claim 44, wherein the medication delivery device comprises an inhaler and the medication delivery data comprises inhalation data.

46. The interactive patient management system of claim 41, wherein the graphic or the audio file comprises a dosage regimen or a reminder message, wherein the dosage regimen or the reminder message is associated with the usage of the medication delivery device.

47. The interactive patient management system of claim 41, wherein the program comprises an instruction for a proper use of the medication delivery device.

48. The interactive patient management system of claim 41, wherein the interface device is further configured to receive the medication delivery data in real-time, determine the program in real-time, incorporate the medication delivery data in the program in real-time, and send the control message to the electronic device in real-time.

49. The interactive patient management system of claim 48, wherein the program comprises a game that engages a patient who uses the medication delivery device.
